(19) United States
(12) Patent Application Publication
(10) Pub. No.: US 2021/0062178 A1
(43) Pub. Date: Mar. 4, 2021

(54) NOVEL XYLOSE ISOMERASE GENE AND POLYPEPTIDE AND USES THEREOF

(71) Applicants: The Regents of the University of California, Oakland, CA (US); Universidade do Minho, Braga (PT)

(72) Inventors: JAVIER A. CEJA-NAVARRO, Richmond, CA (US); PAULO CÉSAR FERNANDES DA SILVA, Amares (PT); BJÖRN FREDRIK JOHAANSSON, Gualtar (PT); EOIN L. BRODIE, Piedmont, CA (US)

(21) Appl. No.: 16/907,108

(22) Filed: Jun. 19, 2020

(30) Foreign Application Priority Data
Jun. 21, 2019 (PT) 20191000033746

(51) Int. Cl. C12N 9/92; C12N 15/74

(52) U.S. Cl. CPC C12N 9/92; C12N 2800/22; C12Y 503/01005; C12N 15/746

(57) ABSTRACT

The present invention provides for a novel D-xylose isomerase (XI) gene that is suitable for metabolic engineering of Saccharomyces cerevisiae for an improved consumption of D-xylose.

### STATEMENT OF GOVERNMENTAL SUPPORT

The invention was made with government support under Contract Nos. DE-AC02-05CH11231 awarded by the U.S. Department of Energy. The government has certain rights in the invention.

### RELATED PATENT APPLICATIONS

The application claims priority to Portuguese Patent Application No. 20191000033746, filed Jun. 21, 2019, which is herein incorporated by reference in its entirety.

### FIELD OF THE INVENTION

The present invention is in the field of genetics, namely in the field of genetic and metabolic engineering.

### BACKGROUND OF THE INVENTION

The yeast Saccharomyces cerevisiae is the organism of choice for industrial production of ethanol. This is essentially due to its high ethanol tolerance and the ability to ferment under strictly anaerobic conditions. Additionally, unlike its prokaryotic counterparts, S. cerevisiae withstands low pH and is insensitive to bacteriophage infection, which is particularly relevant in large industrial processes (Moysés et al, Int. J. Mol-Sci. 2016, 17, 207).

Carbohydrate rich substrates such as lignocellulosic hydrolysates remain one of the primary sources of potentially renewable fuel and bulk chemicals. The pentose sugar D-xylose is often present in significant amounts along with hexoses such as glucose and galactose. For low value/high volume products, yield is of paramount importance for process economy. The preferred industrial organism Saccharomyces cerevisiae can acquire the ability to metabolize D-xylose through expression of heterologous Xylose Isomerase (XI). This enzyme is notoriously difficult to express in S. cerevisiae and only thirteen genes have been reported to be active.

Lignocellulosic material continues to be the most promising renewable raw material for the production of sustainable fuels and fine chemicals. Xylan is the second most abundant biopolymer on earth, which contains mostly the pentose sugar D-xylose. Baker's yeast or Saccharomyces cerevisiae is the preferred organism for industrial transformation of sugars derived from lignocellulose due to innate resistance to fermentation inhibitors. Expression of heterologous pathways are necessary for D-xylose as it is not metabolized naturally by S. cerevisiae. D-xylose metabolism remains a metabolic bottleneck in S. cerevisiae despite the development of several types of pathways for the consumption of this sugar.

D-xylose metabolic pathways can be classified into two main categories: xylose reductase-xylitol dehydrogenase (XR-XDH) and xylose isomerase (XI). The XR-XDH pathway converts D-xylose to xylitol by reduction with NADPH or NADH followed by an oxidation with NAD+ to Xylulose in an overall redox neutral process. Alternatively, the same reaction is carried out by a single XI enzyme without co-factors. The XR-XDH pathway is mainly found in fungi while the XI pathway is common in prokaryotes. The currently most promising D-xylose metabolic pathways are based on the prokaryotic xylose isomerase route. The reason for this is that although the overall reaction is redox neutral, the D-xylose reductase/Xylitol dehydrogenase pathway suffers from a NAD(P)H cofactor imbalance that has proven hard to remedy. However, the xylose isomerase pathway suffers from low capacity and inhibition by xylitol in particular (Brat et al. 2009). Another issue is that the XI is rather difficult to express. Several unsuccessful attempts have been made to express XIs, such as the ones from Escherichia coli (Briggs et al. 1984; Sarthy et al. 1987), Bacillus subtilis, Actinoplanes missouriensis (Amore et al. 1989), Lactobacillus pentosus (Hallborn 1995) and Clostridium thermosulfurogenes (Moes et al. 1996). The first successfully expressed XI was a thermostable enzyme from Thermus thermophilus (Walfridsson et al. 1996) followed by a fungal XI from Piromyces spp (Kuyper et al. 2004). The recombinant strain showed considerably high XI activity of 1.1 U·mg⁻¹, but still low growth rates in xylose under aerobic conditions and no growth in anaerobiosis. Prolonged adaptation in xylose under anaerobic conditions resulted in the isolation of a strain (RWB202-AFX), which showed a specific growth rate of 0.03 h⁻¹ and ethanol yield of 0.42 g·g⁻¹ (Moysés et al, 2016).

Thirteen different xylose isomerases have been reported to actively express in S. cerevisiae to date (Table 1). Interestingly, the two eukaryotic xylose isomerases in Table 1 (entry #2 and #3) come from the same division (Neocallimastigomycota). These fungi are known for possessing genes that are originated from lateral gene transfer from bacteria and their xylose isomerases are of prokaryotic origin and have been taken up recently in evolutionary terms.

TABLE 1
Xylose Isomerase genes expressed in Saccharomyces cerevisiae.

| # | Source | Type | Reference |
|---|---|---|---|
| 1 | Thermus | Prokaryote, | (Walfridsson et al. 1996; |
| | thermophilus | Gram− | Gárdonyi and Hahn- |
| | | | Hägerdal 2003) |
| 2 | Piromyces sp. E2 | Eukaryote | (Kuyper et al. 2003) |
| 3 | Orpinomyces | Eukaryote | (Madhavan et al. 2009) |
| 4 | Clostridium | Prokaryote, | (Brat et al. 2009; |
| | phytofermentans | Gram+ | Seike et al. 2019) |
| 5 | Soil (unspecified) | Prokaryote | (Parachin and Gorwa- |
| | | | Grauslund 2011) |
| 6 | Bacteroides | Prokaryote, | (Ha et al. 2011) |
| | stercoris | Gram− | |
| 7 | Ruminococcus | Prokaryote, | (Aeling et al. 2012) |
| | flavefaciens | Gram+ | |
| 8 | Prevotella | Prokaryote, | (Hector et al. 2013) |
| | ruminicola | Gram− | |
| 9 | Burkholderia | Prokaryote, | (de Figueiredo |
| | cenocepacia | Gram− | Vilela et al. 2013) |
| 10 | Clostridium | Prokaryote, | (Ota et al. 2013) |
| | cellulovorans | Gram− | |
| 11 | Bacteroides | Prokaryote, | (Peng et al. 2015) |
| | vulgatus | Gram− | |
| 12 | Bovine rumen | Eukaryote | (Hou et al. 2016) |
| | (unspecified) | | |
| 13 | Termite gut | Prokaryote | (Katahira et al. 2017) |

There are three eukaryotic isomerases which have been isolated from ruminant animals which may imply adaptation to a temperature of around 37° C. Interestingly, there are only two reports of xylose isomerases isolated from metagenomes and subsequently expressed in S. cerevisiae (Table 1, #12 and #13). A xylose isomerase was amplified using degenerate primers from bovine rumen contents using degenerate PCR primers for conserved XI specific sequences (Hou et al. 2016). Another was identified using a similar PCR based technique from protists residing in the hindgut of the termite Reticulitermes speratus (Katahira et al. 2017). An alternative way of identifying xylose isomerases is through the assembly of high-throughput metagenomic sequences, in-silico translation followed by BLAST search using known XI sequences as query. A subset of identified genes is then synthesized and in-vitro optimized for a specific host. This strategy would do away with the unpredictable and potentially biased use of PCR with degenerate primers. Potential hurdles would be the fidelity with which the genes are assembled and possible divergence of the genetic code usage in the metagenomic data.

In the present work, a cluster of XI genes were identified using this method, three of which were synthesized. One of the three sequences expressed actively in S. cerevisiae, proving that this strategy, amenable to high-throughput analysis, is a viable option for the identification of novel XI genes for expression in S. cerevisiae. The newly identified enzyme enables yeast to proliferate in a xylose containing medium as the sole carbon source at the highest growth rate reported so far in strains not adapted to the carbon source.

### SUMMARY OF THE INVENTION

The present invention provides for an isolated or purified D-xylose isomerase (XI) having a maximal velocity equal to or more than about three times that of Piromyces XI, or any one of the XI comprising SEQ ID NO:3-6.

In some embodiments, the XI has an amino acid sequence having at least 80% sequence identity with SEQ ID NO:2. In some embodiments, the XI has an amino acid sequence having at least 85% sequence identity with SEQ ID NO:2. In some embodiments, the XI has an amino acid sequence having at least 90% sequence identity with SEQ ID NO:2. In some embodiments, the XI has an amino acid sequence having at least 95% sequence identity with SEQ ID NO:2. In some embodiments, the XI has an amino acid sequence having at least 99% sequence identity with SEQ ID NO:2. In some embodiments, the XI has an amino acid sequence comprising SEQ ID NO:2. In some embodiments, the XI comprises the indicated conserved amino acid residues shown in FIG. 3.

The present invention provides for a nucleic acid comprising an open reading frame (ORF) encoding the XI of the present invention. In some embodiments, the ORF is codon optimized for a microbe. In some embodiments, the microbe is one described herein. In some embodiments, the ORF is codon optimized for expression in a Sacchromyces species. In some embodiments, the ORF is codon optimized for expression in Sacchromyces cerevisae. In some embodiments, the ORF comprises a nucleotide sequence of SEQ ID NO:1. In some embodiments, the nucleic acid is double-or single-stranded DNA or RNA.

The present invention provides for a vector comprising the nucleic acid of the present invention. In some embodiments, the ORF is operatively linked to a promoter capable of expressing the ORF, such as in an in vitro or in vivo system. In some embodiments, the vector comprises one or more nucleotides sequences which confers stable residence or replication in a microbe, such a microbe described herein. In some embodiments, the vector is a plasmid. In some embodiments, the vector is an expression vector. In some embodiments, the ORF further encodes a nucleotide sequence encoding an amino acid sequence tag that specifically binds to, or has a high affinity, for a metal ion, a specific peptide (such as the binding region of antibody), or any other compound. In some embodiments, the amino acid sequence tag is a polyhistidine tag. In some embodiments, the amino acid sequence tag does not interfere with or reduce the enzymatic activity and/or maximal velocity of the XI.

The present invention provides for a host cell comprising the vector of the present invention. The host cell can be any microbe described herein. In some embodiments, the host cell is capable of expressing the XI.

The present invention provides for a method for constructing a vector of the present invention, the method comprising: introducing the ORF of XI of the present invention into a vector to produce the vector of the present invention.

The present invention provides for a method for producing the XI of the present invention, the method comprising: (a) optionally providing a vector of the present invention, (b) introducing the vector into a host cell, (c) optionally culturing or growing the host cell in a culture medium such that the host cell expresses the XI, and (d) optionally separating the XI from the rest of the host cell.

The present invention provides for a method for treating a biomass, the method comprising: providing a composition comprising a biomass and an isolated or purified XI of the present invention. In some embodiments, the providing step comprises introducing the isolated or purified XI to the biomass or mixing the biomass and the isolated or purified XI.

A new D-xylose isomerase was cloned from microorganisms in the gut of Odontotaenius disjunctus. Expression of the new XI enzyme results in a considerably faster aerobic growth of S. cerevisiae with D-xylose as the sole carbon source. Maximal velocity of the new enzyme is at least three times higher than the one measured with the Piromyces enzyme. The new XI is a useful addition to the molecular toolbox for genetic modification of S. cerevisiae for the metabolism of second-generation substrates.

An XI sequence from the gut of Odontotaenius disjunctus, a wood-feeding beetle, was identified through analysis of genes present in metagenome assemblies with XI functional predictions. Although homologous to the XI from Piromyces sp. metagenome scaffold gene neighborhoods and metagenome binning identified the gene as being of bacterial in origin and the host as a probable Clostridium species. The new XI enzyme shares 89% identity with XI enzyme from Porphyromonadaceae bacterium (accession no. HCC52362), and 82% identity with XI enzyme from Bacteroides stercoris (accession no. WP_034536238) which has been successfully expressed in Saccharomyces cerevisiae.

Screening of candidates was performed by scoring growth of clones carrying a library plasmid containing a XI gene on solid media with D-xylose as the sole, or main, carbon source. The clones expressed an incomplete D-xylose metabolic pathway in addition to the XI.

The clone that showed the best performance on solid medium was the one expressing XI identified as “8454_2”. This clone was then was cultivated in liquid medium containing xylose as the sole carbon source in parallel with an identical clone carrying the same metabolic pathway, but instead expressing a XI from Piromyces sp (opt.PiXI). Opt.PiXI is a codon-optimized version of the XI gene from Piromyces sp.

The Saccharomyces cerevisiae strains and plasmids used in this work are listed in Table 2. Yeast strains were cultivated in complex media containing 2% (w/v) bacto-peptone (BD biosciences, San Jose, Calif., USA), 1% (w/v) yeast extract with 2% (w/v) glucose (YPD), 2% (w/v) maltose (YPM), or 2% (w/v) xylose (YPX); or in defined synthetic complete media (SC) lacking specific amino acids for selection, containing 0.67% (w/v) yeast nitrogen base without amino acids (BD, Franklin Lakes, N.J., USA), 0.07% amino acid dropout mix (minus HULT: His, Ura, Leu, and Trp), 50 mM potassium hydrogen phthalate, 2% (w/v) glucose, 2% (w/v) maltose (SCm) or 2% (w/v) xylose (SCx). SC media had pH values adjusted to 5.5. Amino acids were added as required to a concentration of 0.008% (w/v) histidine, uracil and tryptophan, and 0.02% (w/v) leucine. Plates were incubated at 30° C. and liquid cultures were further grown on an orbital shaker at 200 revolutions/minute (rpm).

TABLE 2
Saccharomyces cerevisiae strains and plasmids used in this work.

| Strain | Relevant genotype | Reference |
|---|---|---|
| EBY.VW4000 | MATa ura3-52 his3-Δ1 | (Wieczorke et al. 1999) |
| | leu2-3,112 trp1-289 | |
| CEN.PK111-61A | Matalpha MATalfa ura3-52 | (Entian and Kötter 2007) |
| | his3-Δ1 leu2-3,112 TRP1 | |
| Plasmid | Relevant features | Reference |
| pYPK0_XTTRRG | URA3; XKS1, TAL1, TKL1, | Present disclosure |
| | RPE1, RKI1, Gxf1 | |
| pLBL3 | LEU2 | Present disclosure |
| pLBL3_8454_2 | LEU2; 8454_2 xylose | Present disclosure |
| | isomerase | |
| pLBL3_opt_PiXI | LEU2; optimized xylose | Present disclosure |
| | isomerase | |

Specific enzymatic activity was measured using a coupled enzyme (sorbitol dehydrogenase—SDH) that converts the product of XI (xylulose) into xylitol. In this process, for each molecule of xylose converted to xylulose, a molecule of NADH is converted in NAD+. NADH depletion is quantified by spectrophotometry at an optical density of 340 nm, and XI activity is stoichiometrically inferred. For the enzymatic activity, crude cell extracts were prepared using the same conditions for both strains (carrying 8454_2 or opt.PiXI genes) and immediately used.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Growth rates of Saccharomyces cerevisiae cultures in defined media containing xylose (20 g/L) as the sole carbon source. Data points represent an average of at least 3 biological replicates with standard error of the mean indicated.

FIG. 2. Specific enzymatic activity of Xylose Isomerase enzymes 8454_2 and opt.PiXI (codon optimized Piromyces sp. XI gene). Data points represent an average of at least 3 biological replicates with standard error of the mean indicated.

FIG. 3. The amino acid sequence of SEQ IDNO:2 is compared to the amino acid sequences of Piromyces species xylose isomerase encoded by xy1A (Accession No. Q9P8C9; SEQ ID NO:5). Residues underlined are believed to form a coiled coil structure. Residues indicated by a bar are conserved. Residues indicated by an asterisk are conserved and are believed to contain eight manganese (Mn²⁺) ligands possibly involved in regulating the catalytic activity of XI.

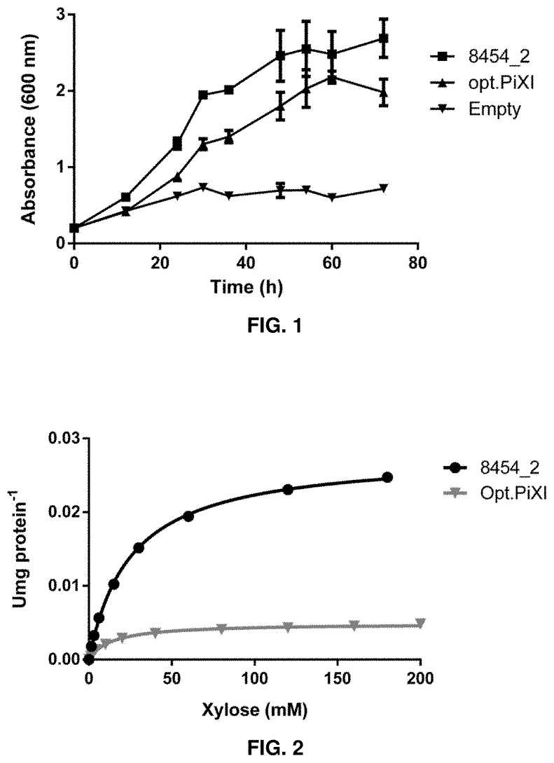

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Carbohydrate rich substrates such as lignocellulosic hydrolysates remain one of the primary sources of potentially renewable fuel and bulk chemicals. The pentose sugar D-xylose is often present in significant amounts along with hexoses such as glucose and galactose. For low value/high volume products, yield is of paramount importance for process economy. In one particular industrial organism Saccharomyces cerevisiae can acquire the ability to metabolize D-xylose through expression of heterologous xylose isomerase (XI). This enzyme is notoriously difficult to express in S. cerevisiae and so far only thirteen genes have been reported to be active. A novel D-xylose isomerase is synthesized and cloned from microorganisms in the gut of Odontotaenius disjunctus, a wood-feeding beetle, that is identified through analysis of genes present in metagenome assemblies with XI functional predictions. Although sharing 79% homology with the XI from Piromyces sp., metagenome scaffold gene neighborhoods and metagenome binning identified the gene as bacterial in origin and the host as a Clostridium species. Expression of the new XI enzyme results in faster aerobic growth of S. cerevisiae with D-xylose as the sole carbon source. Maximal velocity of the new enzyme is three times higher than the one measured with the Piromyces sp. enzyme. In some embodiments, the new XI is a useful addition to the molecular toolbox for genetic modification of S. cerevisiae for the metabolism of second-generation substrates. The new XI exhibits a Km for D-xylose of 19 mM and three times higher isomerization maximal velocity (Vmax) than the XI from Piromyces sp. under identical biological backgrounds and experimental conditions.

The present invention provides for:

An isolated or synthesized polypeptide comprising an amino acid sequence at least 95% identical to a sequence from a list consisting of: SEQ ID NO: 2, as a yeast growth enhancer.

An isolated or synthesized polynucleotide encoding a polypeptide according to the isolated or synthesized polypeptide of the present invention, wherein the polynucleotide comprises a nucleotide sequence at least 95% identical to SEQ ID NO: 1, as a yeast growth enhancer.

The polynucleotide of the present invention, wherein the polynucleotide is a deoxyribonucleic acid or a ribonucleic acid molecule, namely mRNA, tRNA or rRNA molecule.

The polypeptide or polynucleotide of the present invention wherein the Saccharomyces growth enhancer is a Saccharomyces cerevisiae growth enhancer.

The polypeptide or polynucleotide of the present invention wherein said amino acid or nucleotide sequence, respectively, is 96%, 97%, 98% or 99% identical to said SEQ ID NO:1, SEQ ID NO:2, or mixtures thereof.

The polypeptide or polynucleotide of the present invention wherein said amino acid or nucleotide sequence, respectively, is 100% identical to said SEQ ID NO:1, SEQ ID NO:2.

Protein of the present invention, comprising the amino acid sequence is SEQ ID NO:2.

A composition comprising at least one sequence 95% identical to the sequence from a list consisting of: SEQ ID NO:1, SEQ ID NO:2, or mixtures thereof.

Vector comprising the DNA sequence of the present invention.

Plasmid comprising the vector of the present invention.

Host cell comprising an expression vector or the plasmid of the present invention, wherein the host cell is a yeast.

Saccharomyces, such as Saccharomyces cerevisiae, comprising an expression vector or the plasmid of the present invention.

Use of the polypeptide of the present invention as a metabolism booster, particularly by accelerating the growth of Saccharomyces cerevisiae.

Use of the Saccharomyces of the present invention as a fermentation improver or a bakery improver, such as a D-xylose consumption improver.

Use of the Saccharomyces of the present invention in the production of biofuel.

A nucleotide sequence encoding SEQ ID NO:2 is as follows:

(SEQ ID NO: 1)
ATGACATACTTTCCCACAGTGGAGAAGATAAAATTTGAGGGCAAGGAGTC
CAAGAATCCGCTGGCGTTTAGGTATTACGACCCCGAGAAGATGGTCTACG
GTAAAAAGATGAAGGACTGGTTCAAATTTTCCATGGCCTGGTGGCATACT
TTGTGTGCGGAGGGCGGCGACCCTTTCGGTGGGGGTACAAAAACGTTCCC
ATGGGCACAAGGTAGCTCTGCCTTAGAGGTGGCGAAACAGCGTCTGGATG
CCGGCTTTGAGTTTATGCAGAAAATAGGCATCGAGTATTACTGCTTCCAC
GATATTGATTTGATCTCAGAAGGTGATAGTATCGAGGAATACGAAAGCAA
CCTGAAAGCGATTGTGGCATACGCTAAGCAAAAACAAGCGGAAACGGGAA
TAAAGCTTCTATGGGGCACAGCGAACGTTTTCAGTCATAAAAGGTACATG
AACGGGGCCGCGACAAACCCGGACTTCGAAGTGGTTAGCAGGGCAGCGCT
ACAGATAAAGAATGCAATTGACGCGACCATCGAGTTGGGTGGTGAGAACT
ACGTCTTCTGGGGAGGTAGAGAGGGGTACTCTTCCCTGCTTAACACAGAG
ATGAAGAGAGAAAAAGATCATCTTGCGACCATCTTAACCAAGGCAAGAGA
TTATGCTCGTAGTAAAGGCTTTAAGGGCAACTTTCTAATTGAACCAAAAC
CTATGGAGCCCACCAAACATCAATACGATGTCGATACAGAAACGGTGATA
GGATTTTTAAGGGCGCATGGTCTGGATAAGGATTTTAAAGTGAACATAGA
AGTTAATCACGCAACGTTGGCTGGGCATACCTTTGAGCATGAATTGCAAT
GCGCCGTAGACGCCGGCATGCTAGGCTCAATAGACGCGAACAGGGGGGAC
TATCAAAATGGTTGGGACACAGATCAGTTCCCCGTTGATGTGAATGAACT
TACTCAAGCGATGCTTGTAATTTTGAAGGGCGGCGGCTTGCAGGGTGGTG
GTACTAATTTCGATGCGAAGACAAGGCGTAACTCCACAGACTTAGAAGAT
ATTTTTATTGCGCATATAGCCGGAATGGATACTTTCGCCCGTGCACTTGA
GTCTGCGGCAGCGCTATTGGAAGACTCTCCGTACGAGAAGATGTTAAAGG
ACAGATATGCGTCATTCGATGCCGGTAAAGGCAAGGAGTTTGAAGATGGG
AAGCTATCACTTGAAGACATTGTAGCATATGCGAAGTCCAAAGGGGGCGA
ACCGGCCCAAATCAGTGGTAAACAGGAGCTGTATGAGGCGCTGGTAAACA
TGTATATCTAA.

An amino acid sequence of the XI of the present invention is as follows:

(SEQ ID NO: 2)
MTYFPTVEKIKFEGKESKNPLAFRYYDPEKMVYGKKMKDWFKFSMAWWHT
LCAEGGDPFGGGTKTFPWAQGSSALEVAKQRLDAGFEFMQKIGIEYYCFH
DIDLISEGDSIEEYESNLKAIVAYAKQKQAETGIKLLWGTANVFSHKRYM
NGAATNPDFEVVSRAALQIKNAIDATIELGGENYVFWGGREGYSSLLNTE
MKREKDHLATILTKARDYARSKGFKGNFLIEPKPMEPTKHQYDVDTETVI
GFLRAHGLDKDFKVNIEVNHATLAGHTFEHELQCAVDAGMLGSIDANRGD
YQNGWDTDQFPVDVNELTQAMLVILKGGGLQGGGTNFDAKTRRNSTDLED
IFIAHIAGMDTFARALESAAALLEDSPYEKMLKDRYASFDAGKGKEFEDG
KLSLEDIVAYAKSKGGEPAQISGKQELYEALVNMYI.

The amino acid sequence of Bacteroides stercoris xylose isomerase is as follows:

(SEQ ID NO: 3)
MATKEYFPGIGKIKFEGKESKNPMAFRYYDAEKVIMGKKMKDWLKFSMAW
WHTLCAEGGDQFGGGTKHFPWNGDADKLQAAKNKMDAGFEFMQKMGIEYY
CFHDVDLCDEADTIEEYEANLKAIVAYAKQKQEETGIKLLWGTANVFGHA
RYMNGAATNPDFDVVARAAVQIKNAIDATIELGGSNYVFWGGREGYMSLL
NTDQKREKEHLAQMLTIARDYARARGFKGTFLIEPKPMEPTKHQYDVDTE
TVVGFLKAHGLDKDFKVNIEVNHATLAGHTFEHELAVAVDNGMLGSIDAN
RGDYQNGWDTDQFPIDNYELTQAMMQIIRNGGFGDGGTNFDAKTRRNSTD
LEDIFIAHIAGMDVMARALESAAKLLEESPYKKMLADRYASFDSGKGKEF
EEGKLTLEDVVAYAKANGEPKQTSGKQELYEAIVNMYC.

The amino acid sequence of Porphyromonadaceae bacterium xylose isomerase is as follows:

(SEQ ID NO: 4)
MATKTYFPTVEKIKFEGKESKNPLAFRYYDPEKVVYGKKMKEWFKFSMAW
WHTLCAEGGDPFGGGTKTFPWTDGNSALEIAKQRMDAGFEFMQKIGIEYY
CFHDIDLIDEGGSIEEYEANLKAIVAYAKQKQEETGIKLLWGTANVFGHK
RYMNGAATNPDFDVVARAAVQIKNAIDATIELGGENYVFWGGREGYSSLL
NTDMKREKEHLAAMLKAARDYARSKGFNGTFLIEPKPMEPTKHQYDVDAE
TVIGFLRAHGLDKDFKLNIEVNHATLAGHTFEHELQCAADAGLLGSIDAN
RGDYQNGWDTDQFPIDVNELTQAMLVILKSGGLQGGGTNFDAKTRRNSTD
PEDIFIAHVAGMDAFARALEVAAAILENSPYQGMIQNRYASFDAGKGKEF
EQGQLSLEDLVAYAKQKGEPAQISGKQELYEAIVNMYI.

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. An isolated or purified D-xylose isomerase (XI) having a maximal velocity equal to or more than about three times that of Piromyces XI, or any one of the XI comprising SEQ ID NO:3-5.

2. The isolated or purified XI of claim 1, wherein the XI has an amino acid sequence having at least 80% sequence identity with SEQ ID NO:2.

3. The isolated or purified XI of claim 2, wherein the XI has an amino acid sequence having at least 85% sequence identity with SEQ ID NO:2.

4. The isolated or purified XI of claim 3, wherein the XI has an amino acid sequence having at least 90% sequence identity with SEQ ID NO:2.

5. The isolated or purified XI of claim 4, wherein the XI has an amino acid sequence having at least 95% sequence identity with SEQ ID NO:2.

6. The isolated or purified XI of claim 5, wherein the XI has an amino acid sequence having at least 99% sequence identity with SEQ ID NO:2.

7. The isolated or purified XI of claim 6, wherein the XI has an amino acid sequence comprising SEQ ID NO:2.

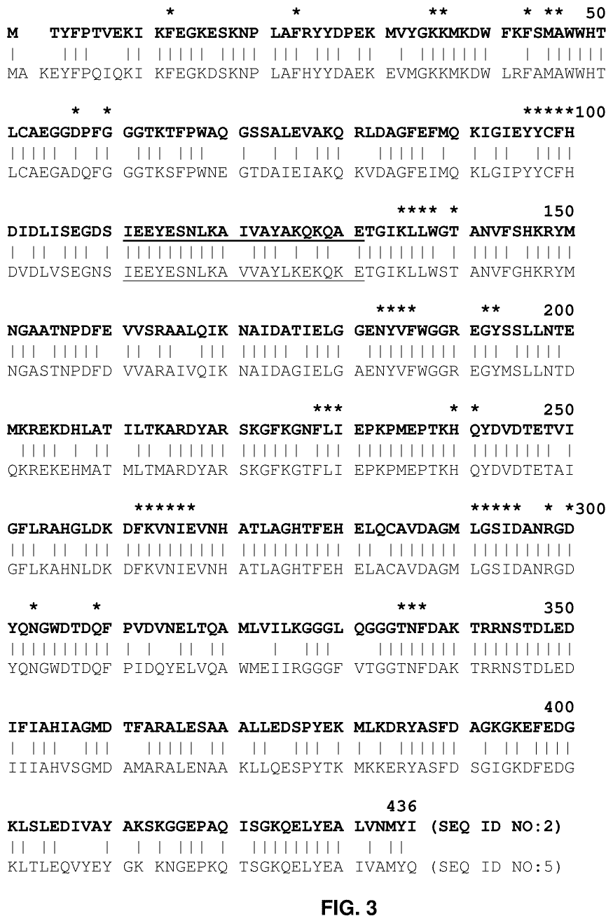

8. The isolated or purified XI of claim 2, wherein the XI comprises the indicated conserved amino acid residues shown in FIG. 3 with an asterisk or a bar.

9. A nucleic acid comprising an open reading frame (ORF) encoding the XI of claim 1.

10. The nucleic acid of claim 9, wherein the ORF is codon optimized for a microbe

11. The nucleic acid of claim 10, wherein the ORF is codon optimized for expression in a Sacchromyces species.

12. The nucleic acid of claim 11, wherein the ORF is codon optimized for expression in Sacchromyces cerevisae.

13. The nucleic acid of claim 12, wherein the ORF comprises a nucleotide sequence of SEQ ID NO:1.

14. A vector comprising the nucleic acid of claim 9.

15. The vector of claim 14, wherein the vector is a plasmid or an expression vector.

16. A host cell comprising the vector of claim 14.

17. The host cell of claim 16, wherein the host cell is a Sacchromyces species.

18. The host cell of claim 17, wherein the host cell is a Sacchromyces cerevisae.

19. A method for producing a D-xylose isomerase (XI), the method comprising: (a) optionally providing a vector of claim 14, (b) introducing the vector into a host cell, (c) optionally culturing or growing the host cell in a culture medium such that the host cell expresses the XI, and (d) optionally separating the XI from the rest of the host cell.

20. A method for treating a biomass, the method comprising: providing a composition comprising a biomass and an isolated or purified XI of claim 1.


SEQUENCE LISTING (5 sequences)

SEQ ID NO: 1 - DNA, 1311 bp, Artificial Sequence
  DNA sequence of the 8454_2 XI
atgacatactttcccacagtggagaagataaaatttgagggcaaggagtccaagaatccgctggcgtttaggtattacgaccccgagaagatggtctacggtaaaaagatgaaggactggttcaaattttccatggcctggtggcatactttgtgtgcggagggcggcgaccctttcggtgggggtacaaaaacgttcccatgggcacaaggtagctctgccttagaggtggcgaaacagcgtctggatgccggctttgagtttatgcagaaaataggcatcgagtattactgcttccacgatattgatttgatctcagaaggtgatagtatcgaggaatacgaaagcaacctgaaagcgattgtggcatacgctaagcaaaaacaagcggaaacgggaataaagcttctatggggcacagcgaacgttttcagtcataaaaggtacatgaacggggccgcgacaaacccggacttcgaagtggttagcagggcagcgctacagataaagaatgcaattgacgcgaccatcgagttgggtggtgagaactacgtcttctggggaggtagagaggggtactcttccctgcttaacacagagatgaagagagaaaaagatcatcttgcgaccatcttaaccaaggcaagagattatgctcgtagtaaaggctttaagggcaactttctaattgaaccaaaacctatggagcccaccaaacatcaatacgatgtcgatacagaaacggtgataggatttttaagggcgcatggtctggataaggattttaaagtgaacatagaagttaatcacgcaacgttggctgggcatacctttgagcatgaattgcaatgcgccgtagacgccggcatgctaggctcaatagacgcgaacaggggggactatcaaaatggttgggacacagatcagttccccgttgatgtgaatgaacttactcaagcgatgcttgtaattttgaagggcggcggcttgcagggtggtggtactaatttcgatgcgaagacaaggcgtaactccacagacttagaagatatttttattgcgcatatagccggaatggatactttcgcccgtgcacttgagtctgcggcagcgctattggaagactctccgtacgagaagatgttaaaggacagatatgcgtcattcgatgccggtaaaggcaaggagtttgaagatgggaagctatcacttgaagacattgtagcatatgcgaagtccaaagggggcgaaccggcccaaatcagtggtaaacaggagctgtatgaggcgctggtaaacatgtatatctaa

SEQ ID NO: 2 - protein, 436 aa, Artificial Sequence
  Amino acid sequence of the 8454_2 XI
MTYFPTVEKIKFEGKESKNPLAFRYYDPEKMVYGKKMKDWFKFSMAWWHTLCAEGGDPFGGGTKTFPWAQGSSALEVAKQRLDAGFEFMQKIGIEYYCFHDIDLISEGDSIEEYESNLKAIVAYAKQKQAETGIKLLWGTANVFSHKRYMNGAATNPDFEVVSRAALQIKNAIDATIELGGENYVFWGGREGYSSLLNTEMKREKDHLATILTKARDYARSKGFKGNFLIEPKPMEPTKHQYDVDTETVIGFLRAHGLDKDFKVNIEVNHATLAGHTFEHELQCAVDAGMLGSIDANRGDYQNGWDTDQFPVDVNELTQAMLVILKGGGLQGGGTNFDAKTRRNSTDLEDIFIAHIAGMDTFARALESAAALLEDSPYEKMLKDRYASFDAGKGKEFEDGKLSLEDIVAYAKSKGGEPAQISGKQELYEALVNMYI

SEQ ID NO: 3 - protein, 438 aa, Bacteroides stercoris
MATKEYFPGIGKIKFEGKESKNPMAFRYYDAEKVIMGKKMKDWLKFSMAWWHTLCAEGGDQFGGGTKHFPWNGDADKLQAAKNKMDAGFEFMQKMGIEYYCFHDVDLCDEADTIEEYEANLKAIVAYAKQKQEETGIKLLWGTANVFGHARYMNGAATNPDFDVVARAAVQIKNAIDATIELGGSNYVFWGGREGYMSLLNTDQKREKEHLAQMLTIARDYARARGFKGTFLIEPKPMEPTKHQYDVDTETVVGFLKAHGLDKDFKVNIEVNHATLAGHTFEHELAVAVDNGMLGSIDANRGDYQNGWDTDQFPIDNYELTQAMMQIIRNGGFGDGGTNFDAKTRRNSTDLEDIFIAHIAGMDVMARALESAAKLLEESPYKKMLADRYASFDSGKGKEFEEGKLTLEDVVAYAKANGEPKQTSGKQELYEAIVNMYC

SEQ ID NO: 4 - protein, 438 aa, Porphyromonadaceae bacterium
MATKTYFPTVEKIKFEGKESKNPLAFRYYDPEKVVYGKKMKEWFKFSMAWWHTLCAEGGDPFGGGTKTFPWTDGNSALEIAKQRMDAGFEFMQKIGIEYYCFHDIDLIDEGGSIEEYEANLKAIVAYAKQKQEETGIKLLWGTANVFGHKRYMNGAATNPDFDVVARAAVQIKNAIDATIELGGENYVFWGGREGYSSLLNTDMKREKEHLAAMLKAARDYARSKGFNGTFLIEPKPMEPTKHQYDVDAETVIGFLRAHGLDKDFKLNIEVNHATLAGHTFEHELQCAADAGLLGSIDANRGDYQNGWDTDQFPIDVNELTQAMLVILKSGGLQGGGTNFDAKTRRNSTDPEDIFIAHVAGMDAFARALEVAAAILENSPYQGMIQNRYASFDAGKGKEFEQGQLSLEDLVAYAKQKGEPAQISGKQELYEAIVNMYI

SEQ ID NO: 5 - protein, 437 aa, Piromyces species
MAKEYFPQIQKIKFEGKDSKNPLAFHYYDAEKEVMGKKMKDWLRFAMAWWHTLCAEGADQFGGGTKSFPWNEGTDAIEIAKQKVDAGFEIMQKLGIPYYCFHDVDLVSEGNSIEEYESNLKAVVAYLKEKQKETGIKLLWSTANVFGHKRYMNGASTNPDFDVVARAIVQIKNAIDAGIELGAENYVFWGGREGYMSLLNTDQKREKEHMATMLTMARDYARSKGFKGTFLIEPKPMEPTKHQYDVDTETAIGFLKAHNLDKDFKVNIEVNHATLAGHTFEHELACAVDAGMLGSIDANRGDYQNGWDTDQFPIDQYELVQAWMEIIRGGGFVTGGTNFDAKTRRNSTDLEDIIIAHVSGMDAMARALENAAKLLQESPYTKMKKERYASFDSGIGKDFEDGKLTLEQVYEYGKKNGEPKQTSGKQELYEAIVAMYQ